(19) United States
(12) Patent Application Publication
(10) Pub. No.: US 2013/0218051 A1
(43) Pub. Date: Aug. 22, 2013

(54) SYSTEMS AND METHODS FOR EVALUATING LIVING TISSUE

(71) Applicant: University of Florida Research Foundation, Inc., (US)

(72) Inventors: Alison Campbell Dunn, Gainesville, FL (US); Daniel John Dickrell, III, Gainesville, FL (US); Wallace Gregory Sawyer, Gainesville, FL (US)

(73) Assignee: UNIVERSITY OF FLORIDA RESEARCH FOUNDATION, INC., Gainesville, FL (US)

(21) Appl. No.: 13/773,185

(22) Filed: Feb. 21, 2013

Related U.S. Application Data
(60) Provisional application No. 61/601,306, filed on Feb. 21, 2012.

(51) Int. Cl. A61B 5/103

(52) U.S. Cl. CPC A61B 5/103
USPC 600/587

(57) ABSTRACT

Disclosed herein is a system for evaluating living tissue comprising a probe device having a probe, an actuator adapted to displace the probe in accordance with a programmed displacement profile, and force sensors that measure deflection of the probe at each position of the probe while the probe is in contact with the tissue. Disclosed herein too is a method comprising contacting a sample with a probe device having a probe, an actuator adapted to displace the probe in accordance with a programmed displacement profile, and force sensors that measure deflection of the probe at each position of the probe while the probe is in contact with the sample; measuring the deflection of the probe at each position of the probe at each point of contact with the sample; and determining whether any tissue present on the sample is a living tissue.

### CROSS-REFERENCE TO RELATED APPLICATIONS

This application claims priority to U.S. Provisional Patent Application No. 61/601,306 filed on Feb. 21, 2012, the entire contents of which are hereby incorporated by reference.

### BACKGROUND

Various devices are currently used to evaluate living tissue. Such devices include tonometry devices, which are typically employed to determine the intraocular pressure of the eye. Some of these devices, such as those used in Goldmann tonometry, are large, expensive pieces of equipment that are relatively complicated to use. More recently, portable tonometry devices have been developed. Although these devices are much more simple in both construction and use, they do not always accurately measure the intraocular pressure because the measurements are dependent upon the way in which the physician or clinician operates the device. It would be desirable to have an alternative apparatus that is both simple and highly accurate. In addition, it would be desirable to have an apparatus that is not limited to use in association with the eye.

### SUMMARY

Disclosed herein is a system for evaluating living tissue comprising a probe device having a probe, an actuator adapted to displace the probe in accordance with a programmed displacement profile, and force sensors that measure deflection of the probe at each position of the probe while the probe is in contact with the tissue.

Disclosed herein too is a method comprising contacting a sample with a probe device having a probe, an actuator adapted to displace the probe in accordance with a programmed displacement profile, and force sensors that measure deflection of the probe at each position of the probe while the probe is in contact with the sample; measuring the deflection of the probe at each position of the probe at each point of contact with the sample; and determining whether any tissue present on the sample is a living tissue.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a schematic view of an embodiment of a system for evaluating living tissue;

FIG. 2 is a block diagram of an example configuration a probe device shown in FIG. 1;

FIG. 3 is a block diagram of an example configuration of a computer shown in FIG. 1;

FIG. 4 is a flow diagram of an embodiment of a method for evaluating living tissue;

FIGS. 5A and 5B illustrate a first example of use of a probe device; and

FIGS. 6A and 6B illustrate a second example of use of a probe device.

### DETAILED DESCRIPTION

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, singular forms like “a,” or “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another elements as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

The term and/or is used herein to mean both “and” as well as “or”. For example, “A and/or B” is construed to mean A, B or A and B.

The transition term “comprising” is inclusive of the transition terms “consisting essentially of” and “consisting of” and can be interchanged for “comprising”.

While this disclosure describes exemplary embodiments, it will be understood by those skilled in the art that various changes can be made and equivalents can be substituted for elements thereof without departing from the scope of the disclosed embodiments. In addition, many modifications can be made to adapt a particular situation or material to the teachings of this disclosure without departing from the essential scope thereof. Therefore, it is intended that this disclosure not be limited to the particular embodiment disclosed as the best mode contemplated for carrying out this disclosure.

Disclosed herein are systems and methods for evaluating living tissue that are simple in both design and use, and highly accurate. In one embodiment, a probe device of the system includes actuators that can be used to contact living tissue and measure forces associated with that contact. In some embodiments, a probe of the device is moved in a direction that is generally perpendicular to the living tissue. In other embodiments, the probe is moved in a direction that is generally parallel to the living tissue. In some embodiments, the forces associated with the contact between the probe and the living tissue are determined at each position of the probe.

In the following disclosure, various specific embodiments are described. It is to be understood that those embodiments are example implementations of the disclosed inventions and that alternative embodiments are possible. All such embodiments are intended to fall within the scope of this disclosure.

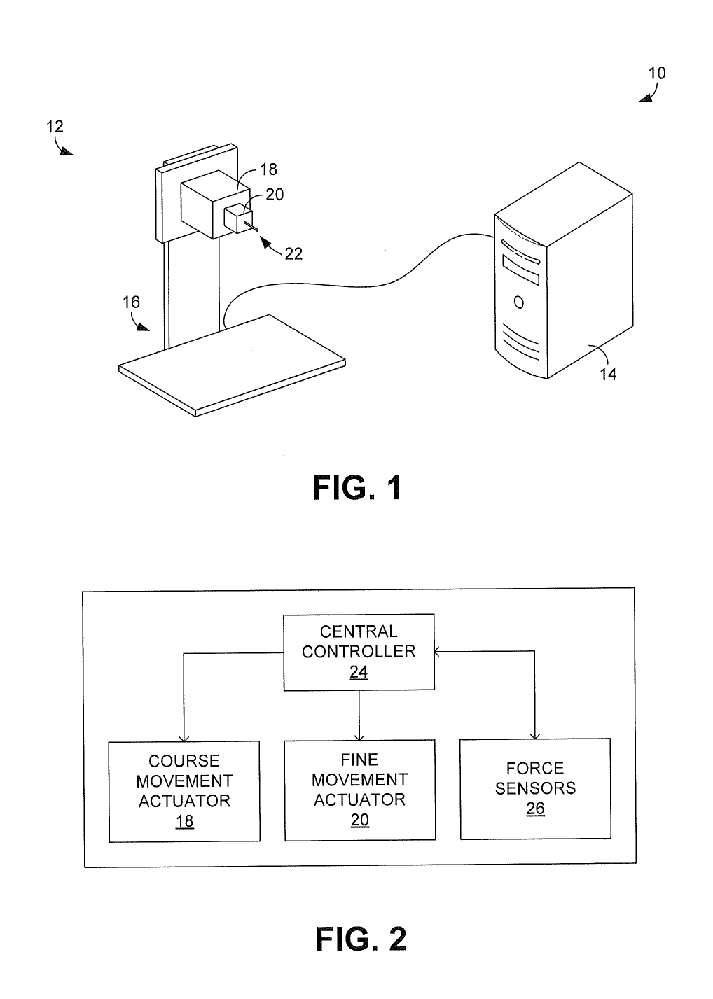

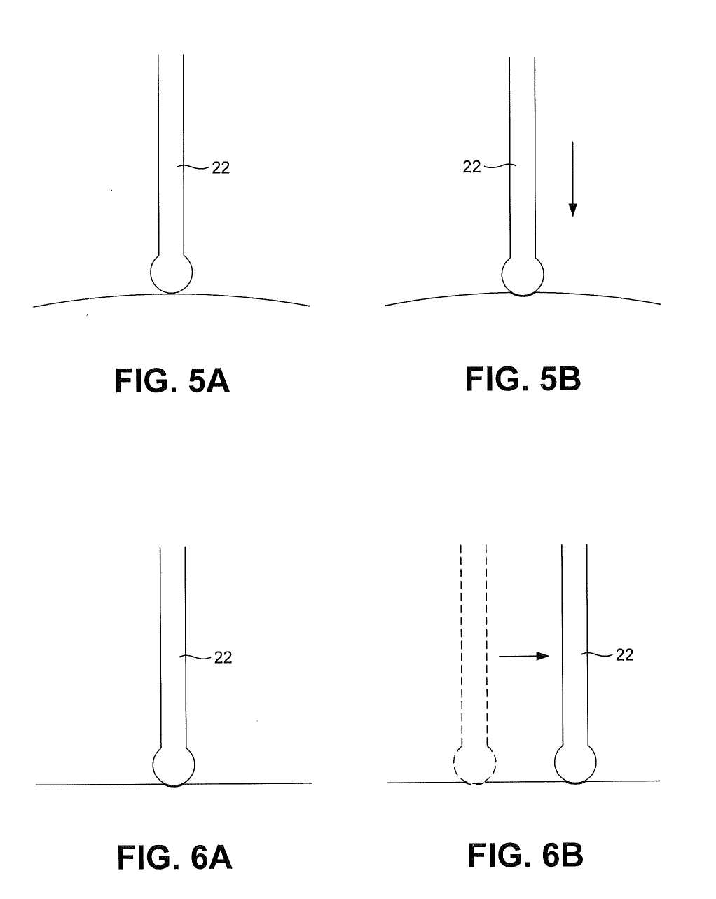

FIG. 1 illustrates an example system 10 for evaluating living tissue. As is shown in FIG. 1, the system 10 generally includes a probe device 12 that is in electrical communication with a computer 14. The probe device 12 comprises a support stand 16 that supports a coarse movement actuator 18 and a fine movement actuator 20. Mounted to the fine movement actuator 20 is a patient interface in the form of a probe 22. In some embodiments, the probe 22 can comprise a glass probe having a bulbous (e.g., round) tip (see, e.g., FIGS. 5 and 6). In such a case, the probe 22 can be assumed to be infinitely stiff and to have substantially no roughness. In other embodiments, the probe 22 can be made of a metal or polymer material.

By way of example, the coarse movement actuator 18 comprises large mechanical micrometer stages or vernier caliper stages and the fine movement actuator 20 comprises piezoelectric stages. The piezoelectric stages can be manufactured from piezo-electric materials that are metal oxides or that are organic polymers.

The metal oxides may include, for example, but is not limited to, lithium niobate (“LiNbO₃”), lithium tantalate (“LiTaO₃”), lithium tetraborate (“Li₂B₄O₇”), barium titanate (“BaTiO₃”), lead zirconate (“PbZrO₃”), lead titanate (“PbTiO₃”), lead zirconate titanate (“PZT”), zinc oxide (“ZnO”), gallium arsenide (“GaAs”), quartz and niobate, berlinite, topaz, tourmaline group materials, potassium niobate, lithium niobate, sodium tungstate, Ba₂NaNb₅O₅, Pb₂KNb₅O₁₅, or the like, or a combination comprising at least one of the foregoing piezoelectric materials.

In another embodiment, the piezoelectric stages may comprise piezoelectric polymers or copolymers or blends comprising at least one piezoelectric polymer. A suitable example of a piezoelectric polymer is polyvinylidene fluoride.

Blends and copolymers of the polyvinylidene fluoride can also be used in the substrate. The copolymers can include block copolymers, alternating block copolymers, random copolymers, random block copolymers, graft copolymers, star block copolymers, or the like, or a combination comprising at least one of the foregoing thermoplastic polymers.

Examples of suitable polymers that can be copolymerized with polyvinylidene fluoride are polytrifluoroethylene, polytetrafluoroethylene, polyacrylamide, polyhexafluoropropylene, polyacrylic acid, poly-(N-isopropylacrylamide), polyacetals, polyolefins, polyacrylics, polycarbonates, polystyrenes, polyesters, polyamides, polyamideimides, polyarylates, polyarylsulfones, polyethersulfones, polyphenylene sulfides, polyvinyl chlorides, polysulfones, polyimides, polyetherimides, polytetrafluoroethylenes, polyetherketones, polyether etherketones, polyether ketone ketones, polybenzoxazoles, polyphthalides, polyacetals, polyanhydrides, polyvinyl ethers, polyvinyl thioethers, polyvinyl alcohols, polyvinyl ketones, polyvinyl halides, polyvinyl nitriles, polyvinyl esters, polysulfonates, polysulfides, polythioesters, polysulfones, polysulfonamides, polyureas, polyphosphazenes, polysilazanes, or the like, or a combination comprising at least one of the foregoing thermoplastic polymers. This list of thermoplastic polymers includes polymers that are electrically insulating. These thermoplastic polymers may be rendered electrically conductive by the addition of intrinsically conductive polymers or electrically conducting fillers to the respective polymers.

Irrespective of their particular construction, the actuators 18, 20 are both configured to displace the probe 22 in the x-, y-, and z-directions.

FIG. 2 illustrates an example configuration for the probe device 12. As is shown in that figure, the probe device 12 includes a central controller 24 that controls the coarse movement actuator 18, the fine movement actuator 20, and force sensors 26. In some embodiments, the force sensors 26 comprise high-resolution capacitive sensors that correlate deflection with force. By way of example, the capacitive sensors are capable of measuring forces as small as approximately 10 to 100 micronewtons (mN). In some embodiments, a first capacitive sensor measures force in a normal direction and a second capacitive sensor measures force in a lateral direction.

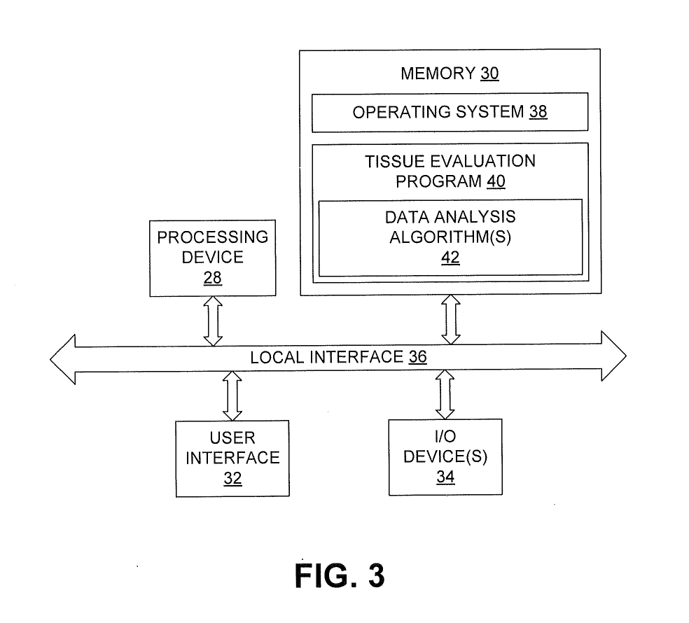

FIG. 3 illustrates an example configuration for the computer 14 shown in FIG. 1. As is shown in FIG. 3, the computer 14 comprises a processing device 28, memory 30, a user interface 32, and at least one I/O device 34, each of which is connected to a local interface 36.

The processing device 28 can include a central processing unit (CPU) or a semiconductor based microprocessor (in the form of a microchip). The memory 30 includes any one of or a combination of volatile memory elements (e.g., RAM) and nonvolatile memory elements (e.g., hard disk, ROM, tape, etc.). The user interface 32 comprises the components with which a user interacts with the computer 14, and the I/O devices 34 are adapted to facilitate communications with other devices, such as the probe device 12.

The memory 30 comprises programs (i.e., logic) including an operating system 38 and a tissue evaluation program 40. In some embodiments, the tissue evaluation program 40 is configured to control operation of the probe device 12, including controlling the probe's direction of displacement and speed of movement. As the probe 22 is contacted with the living tissue, the data that is measured by the probe device 14 is collected and stored by the tissue evaluation program 40. In some embodiments, that data comprises a force-displacement data that identifies the forces measured at each position of the probe 22 while it contacts the living tissue. As is further shown in FIG. 3, the tissue evaluation program 40 can comprise one or more data analysis algorithms 42 that are configured to evaluate the force-displacement data to identify one or more physical properties of the tissue, such as elastic modulus, viscoelastic parameters, plasticity parameters, and the like. In some embodiments, the algorithm(s) 42 can be used to diagnose a condition of the patient, such as a disease.

Although the system for evaluating living tissue has been described above in relation to FIGS. 1-3 as comprising a probe device 12 and a separate computer 14, it is noted that, in some embodiments, the probe device can incorporate the computing functions attributed to the computer in the above description. In such a case, the probe device 12 would comprise a standalone device that is configured to evaluate living tissue. In some embodiments, such a standalone device can be a portable device, such portable tabletop device or a handheld device.

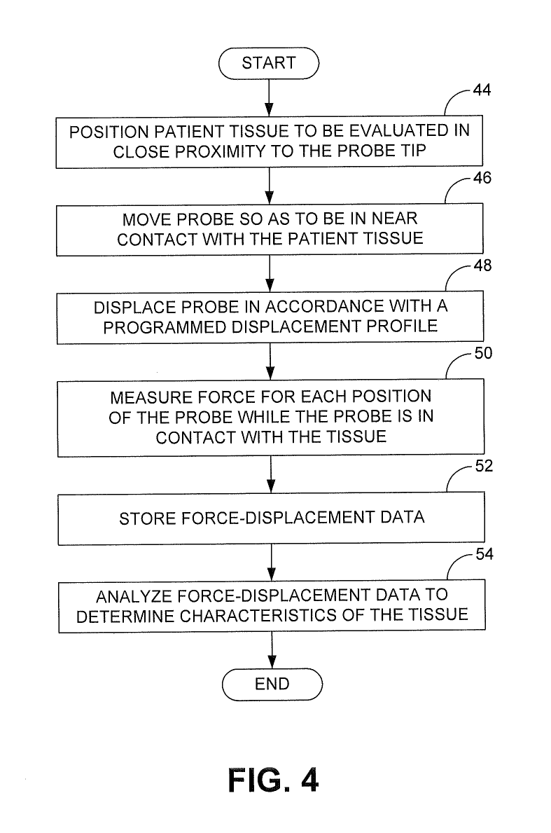

FIG. 4 is a flow diagram of an embodiment of a method for evaluating living tissue. Beginning with block 44, the patient tissue to be evaluated is placed in close proximity with the probe. In some embodiments, alignment apparatus (not shown) can be used to ensure that the patient is positioned correctly. For example, if the tissue is the patient's eye, the patient can place his or her head on a headrest associated with the probe device. Next, the probe is moved so as to be in near contact with the patient tissue, as indicated in block 46. Such movement can be achieved using the coarse movement actuator, the fine movement actuator, or both as necessary.

Referring next to block 48, the probe is placed in contact with the patient tissue in accordance with a programmed displacement profile. Such a profile can dictate the position of the probe tip over time as well as the speed with which the probe is moved. FIGS. 5A and 5B illustrate a first example displacement of the probe 22. In particular, those figures illustrate a micro-indenting procedure in which the probe 22 is extended linearly along its longitudinal axis in a direction that is generally normal to the tissue T so as to indent the tissue. Such a procedure may be useful when the tissue is the eye and the intraocular pressure is to be determined. FIGS. 6A and 6B illustrate a second example displacement of the probe 22. In particular, those figures illustrate a swiping procedure in which the probe 22 is laterally dragged along the surface of the tissue T in a direction generally parallel to the tissue. Such a procedure may be useful when the tissue is the skin and the elasticity of the skin is to be evaluated.

Irrespective of the path that is traveled by the probe, force is measured for each position of the probe while in contact with the tissue, as indicated in block 50, and the force-displacement data is stored, as indicated in block 52. The force-displacement data can then be analyzed to determine characteristics of the tissue, as indicated in block 54. For example, mechanical properties or a condition of the tissue can be determined using one or more algorithms or reference data.

In one embodiment, with reference to the FIG. 1, in one method of using the system a sample (not shown) is brought into contact with the probe 22. The coarse movement actuator 18 and the fine movement actuator 20 are activated to put the probe 22 into appropriate contact with the sample. The probe 22 may be moved in a normal or in a lateral direction relative to the tissue.

The probe 22 may measure properties such as the elastic modulus, flexural modulus, surface resistivity, bulk resistivity, surface roughness, impact toughness, ductility, ultrasound properties, and the like. These properties can be measured against parameters such as temperature, pressure, humidity, ambient conditions, and the like. The resulting measured properties can then be compared against values stored in a database.

In one embodiment, the probe comprises force sensors that include a capacitive sensor that sense deflection of the probe in a normal direction and a capacitive sensor that senses deflection of the probe in a lateral direction. The deflection in the normal direction and the deflection in the lateral direction can be used to estimate and evaluate the type and the quality of the living tissue. The database contains a tissue evaluation program that comprises a data analysis algorithm that evaluates force-displacement data measured by the sensors and determines a physical characteristic of the tissue.

In one embodiment, in one method of manufacturing the system, a stand is disposed on a base plate. Fixedly attached to the stand is a platform which contacts the coarse movement actuator. Affixed to the coarse movement actuator is the fine movement actuator and the probe. The probe, the coarse movement actuator and the fine movement actuator are in electrical communication with a microprocessor (e.g., a computer). The microprocessor is in electrical communication with a database that can store and analyze data collected by the system.

While this disclosure describes exemplary embodiments, it will be understood by those skilled in the art that various changes can be made and equivalents can be substituted for elements thereof without departing from the scope of the disclosed embodiments. In addition, many modifications can be made to adapt a particular situation or material to the teachings of this disclosure without departing from the essential scope thereof. Therefore, it is intended that this disclosure not be limited to the particular embodiment disclosed as the best mode contemplated for carrying out this disclosure.

What is claimed is:

1. A system for evaluating living tissue comprising:
a probe device having
a probe,
an actuator adapted to displace the probe in accordance with a programmed displacement profile, and
force sensors that measure deflection of the probe at each position of the probe while the probe is in contact with the tissue.

2. The system of claim 1, wherein the probe is a glass rod having a round tip.

3. The system of claim 1, wherein the system comprises a coarse movement actuator and a fine movement actuator.

4. The system of claim 3, wherein the coarse movement actuator comprises micrometer stages and the fine movement actuator comprises piezoelectric stages.

5. The system of claim 1, wherein the force sensors are high-resolution capacitive sensors.

6. The system of claim 5, wherein the force sensors include a capacitive sensor that senses deflection of the probe in a normal direction and a capacitive sensor that senses deflection of the probe in a lateral direction.

7. The system of claim 1, wherein the probe is moved in a normal direction relative to the tissue.

8. The system of claim 1, wherein the probe is moved in a lateral direction relative to the tissue.

9. The system of claim 1, further comprising memory that stores a tissue evaluation program that controls movement of the probe.

10. The system of claim 9, wherein the tissue evaluation program comprises a data analysis algorithm that evaluates force-displacement data measured by the sensors and determines a physical characteristic of the tissue.

11. A method comprising:
contacting a sample with a probe device having
a probe,
an actuator adapted to displace the probe in accordance with a programmed displacement profile, and
force sensors that measure deflection of the probe at each position of the probe while the probe is in contact with the sample;
measuring the deflection of the probe at each position of the probe at each point of contact with the sample; and
determining whether any tissue present on the sample is a living tissue.
